(19) United States
(12) Patent Application Publication
(10) Pub. No.: US 2008/0045888 A1
(43) Pub. Date: Feb. 21, 2008

(54) MOBILE BREAST FEEDING ASSEMBLY

(76) Inventors: Michelle Edwards, Owensboro, KY (US); Jessica Gibson, Owensboro, KY (US); Keeley Edwards, Owensboro, KY (US)

Correspondence Address: WHITAKER, CHALK, SWINDLE & SAWYER, LLP, 3500 CITY CENTER TOWER II, 301 COMMERCE STREET, FORT WORTH, TX, 76102-4186, US

(21) Appl. No.: 11/771,322

(22) Filed: Jun. 29, 2007

(30) Foreign Application Priority Data
Aug. 18, 2006 (IN) 1849/DEL/2006

(51) Int. Cl. A61M 1/06

(52) U.S. Cl. 604/76; 604/74

(57) ABSTRACT

A mobile breast-feeding assembly is shown for feeding a child while traveling in a motor vehicle, as where the child being fed is seated in a child safety restraint seat. The apparatus has a conically shaped breast shield, a length of flexible tubing, and a nipple adapter that threadingly engages an exchangeable nipple element. Engagement regions on the nipple adapter allow a variety of exchangeable nipple elements to be utilized with the basic assembly. The assembly can be formed of disposable materials.

### BACKGROUND OF THE INVENTION

1. Field of the Invention

The present invention relates generally to breast feeding assemblies intended for nursing mothers and, more specifically, to a breast feeding assembly which allows a woman to conveniently and safely breast feed a baby in a mobile environment such as when the baby is in a child safety restraint seat while traveling in a moving motor vehicle.

2. Description of the Prior Art

Many mothers, as well as nutritionists, feel that natural breast feeding is the best way to nurture an infant. Supporters claim that breast feeding helps protect infants from various infections, illnesses and allergies among other things. However, breast feeding has long been a struggle for nursing mothers, as there are times where it can be inconvenient or undesirable to breast feed. For example, breast feeding while traveling in a moving motor vehicle is troublesome and can even be dangerous. Injuries sustained from motor vehicle crashes is one of the leading causes of child fatalities. As a result, many states, as a part of their Transportation Codes, require by law that a child be restrained in a child safety seat system during the operation of a motor vehicle. While this certainly adds to the protection of our young, it creates an added inconvenience for nursing mothers that are required to travel extended periods of time with their children. Specifically, nursing has increased travel time in the past, as it was necessary to pull over the vehicle and remove the child from the safety seat during the length of the nursing process. A need exists, therefore, for a breast-feeding assembly that can be used by a nursing mother while traveling in a motor vehicle.

There are a number of prior art patents that generally teach the communication of breast milk from a mother to a nursing child. U.S. Pat. No. 22,018 shows a breast shell which connected by a length of flexible tubing to an artificial nipple. The breast shell (A) is commonly made of glass. The neck (c) seen in FIGS. 1 and 2 is formed with the breast shell for the purpose of facilitating the attachment of the flexible tube (i) to (A). The artificial nipple (o) is connected with the flexible tube (i) by reducing socket (n).

U.S. Pat. No. 420,195 shows a similar design for a nipple shield which is attached by a flexible tube to a rubber nipple (FIG. 2). This reference specifically mentions that one utility of the apparatus is to enable mothers to “empty their breasts when away from their infants, and when traveling and on other occasions . . . ” Note that while FIG. 1 is intended to collect milk in the bottle “A”, the device shown in FIG. 2 is arranged to form a “feeding tube” (column 1, lines 5-7).

U.S. Pat. No. 5,040,756 teaches an apparatus for feeding infants especially adapted for use in vehicles such as automobiles, buses, airplanes or other conveyances. The apparatus comprises a container for a liquid food, such as milk, and a bracket adapted to hold the container and securely support it from the vehicle interior. Two lengths of flexible tubing provide a flow communication from the bottle to a nipple adapted for insertion in the infant's mouth. Although this provides a possible solution to breast feeding while traveling in a motor vehicle, it is somewhat complex and fails to provide a way for breast milk to be communicated from the mother directly to the infant.

Despite the improvements represented by the prior art references discussed above, a need continues to exist for an improved breast-feeding assembly that would safely and effectively allow a nursing mother to breast feed an infant in a child safety restraint seat.

A need also exists to provide a new and improved breast-feeding assembly which is simple in design and economical to manufacture and disposable in nature.

A further need exists to provide such a device which is of durable and reliable construction, and which is easily carried or transported to a location of desired use.

### SUMMARY OF THE INVENTION

The mobile breast-feeding assembly of the invention comprises a breast shield, a length of flexible tubing, a nipple adapter, and an exchangeable nipple element.

The breast shield is conically shaped and has a base, an apex, and an open interior space. A hollowed tip extends from the apex and communicates with the open interior space. The hollowed tip has an inner and outer diameter. The length of flexible tubing has an inner and outer diameter and terminates in first and second opposite extents. The inner diameter of the first extent is sized to matingly receive the outer diameter of the hollowed tip.

The nipple adapter has a closed base and cylindrical sidewalls that extend outwardly from the base to define an open interior. The sidewalls are threaded for matingly engaging an exchangeable nipple element. A hollowed tip extends from the closed base of the nipple adapter and communicates with the open interior. The hollowed tip has an inner and outer diameter. The outer diameter is sized to be matingly received by the inner diameter of the second extent of the flexible tubing. The exchangeable nipple element is capable of engaging the nipple adapter. In the preferred embodiment of the present invention, the threaded sidewalls of the nipple adapter are externally threaded and the exchangeable nipple element is internally threaded to thereby allow mating engagement.

The hollowed tips extending from the breast shield and the nipple adapter have specially formed retention surfaces thereon which permit the flexible tubing to move in one direction onto the hollowed tips while limiting the movement of the flexible tubing in an opposite direction off the hollowed tips. In the preferred embodiment of the present invention, the retention surfaces are a series of barbs.

In the preferred embodiment of the present invention, the mobile breast-feeding assembly described above is adapted for feeding a child while traveling in a motor vehicle. More specifically, the child being fed is seated in a child safety restraint seat.

Additional objects, features and advantages will be apparent in the written description which follows.

### BRIEF DESCRIPTION OF THE DRAWINGS

FIG. 1 provides a perspective view of the mobile breast-feeding assembly of the present invention.

FIG. 2 provides an enlarged view of the breast shield element as shown in FIG. 1.

FIG. 3 provides an enlarged perspective view of the nipple adapter as shown in FIG. 1, showing an exchangeable nipple element in phantom lines.

### DETAILED DESCRIPTION OF THE INVENTION

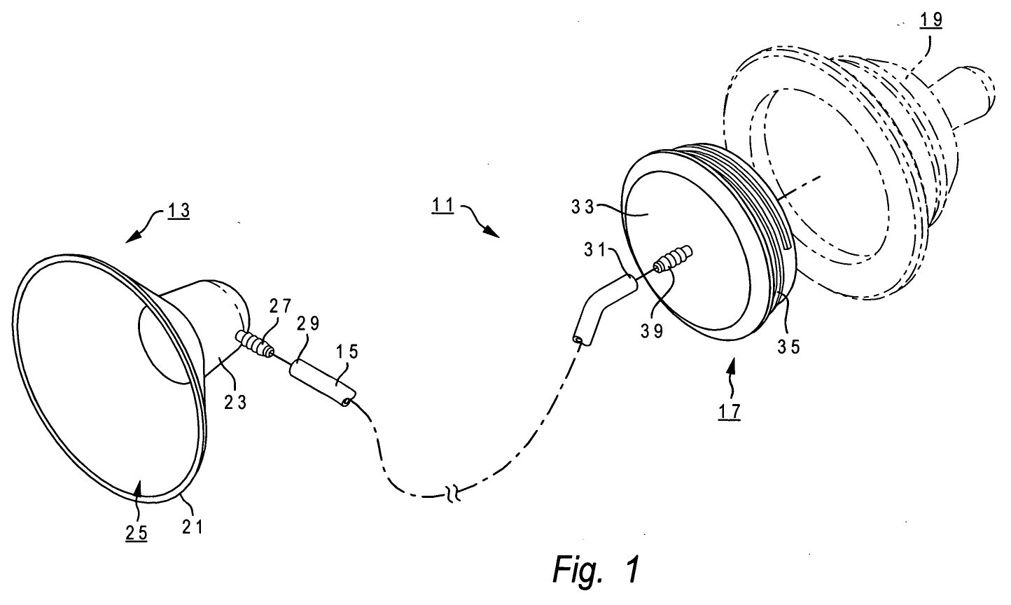

The mobile breast-feeding assembly of the invention will now be described with reference to the accompanying drawing FIGS. 1-3. The embodiments herein and the various features and advantageous details thereof are explained more fully with reference to the non-limiting embodiments that are illustrated in the accompanying drawings and detailed in the following description. Descriptions of well-known components and processes and manufacturing techniques are omitted so as to not unnecessarily obscure the embodiments herein. The examples used herein are intended merely to facilitate an understanding of ways in which the invention herein may be practiced and to further enable those of skill in the art to practice the embodiments herein. Accordingly, the examples should not be construed as limiting the scope of the claimed invention.

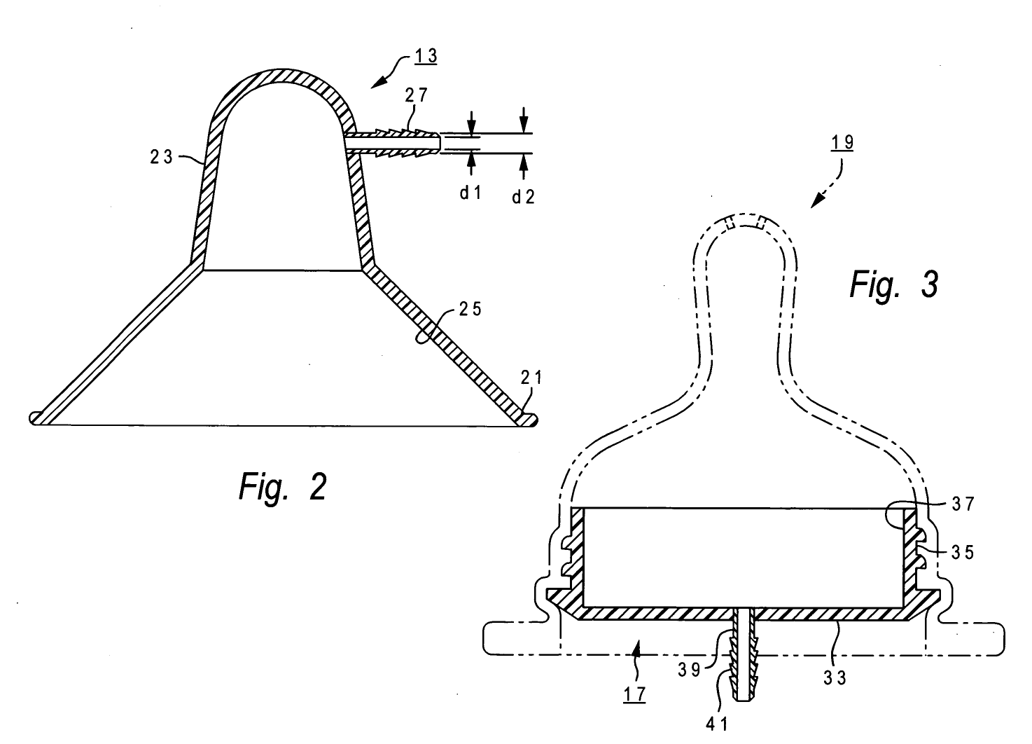

Turning to FIG. 1, there is shown the mobile breast-feeding assembly of the present invention, designated generally as 11. The present invention has as its general purpose to provide an improved breast-feeding assembly that would safely and effectively allow a nursing mother to breast feed an infant in a child safety restraint seat while traveling in a motor vehicle. As seen in FIG. 1, the mobile breast-feeding assembly 11 includes a breast shield 13, a length of flexible tubing 15, a nipple adapter 17 and an exchangeable nipple element 19. As shown in FIGS. 1 and 2, the breast shield 13 is conically shaped, having a base 21, an apex 23, and an open interior space 25. The base 21 has a diameter sufficiently large enough to cover the nipple of the nursing mother. For example, in one embodiment of the present invention, the base of the breast shield has a diameter of approximately 3.125 inches. A hollowed tip 27 extends from the apex 23 of the breast shield 13 and communicates with the open interior space 25. The hollowed tip 27 has an inner and outer diameter (illustrated as “d₁” and “d₂” in FIG. 2). The inner diameter of the hollowed tip is large enough to allow liquid to freely pass from the open interior space 25 of the breast shield.

As also shown in FIG. 1, a length of flexible tubing 15 connects the breast shield 13 and the nipple adapter 17 in use. The flexible tubing 15 has an inner and outer diameter, and terminates in first and second opposite extents 29, 31. The inner diameter of the first extent 29 is sized to matingly receive the outer diameter of the hollowed tip 27 of the breast shield 13. In this manner, any liquid that passes through the hollowed tip 27 is thereby communicated with the interior of the flexible tubing 15. Furthermore, liquid introduced at the first extent of the flexible tubing 15 will thereby flow along the interior towards the second opposite extent 31. The flexibility of the tubing allows any number of arrangements between the locations of the mother and child relative to one another during the nursing procedure.

As best seen in FIGS. 1 and 3, the nipple adapter has a closed base 33 and cylindrical sidewalls 35 that extend outwardly from the base to define an open interior 37. The sidewalls 35 have engagement surfaces formed thereon for matingly engaging the exchangeable nipple element 19. For example, the cylindrical sidewalls 35 can be externally threaded for engaging mating internal threads on the exchangeable nipple element 19. In the preferred embodiment of the present invention, the threaded sidewalls of the nipple adapter are externally threaded and the exchangeable nipple element is internally threaded. It will be understood by those skilled in the art that other types of engagement surfaces might also be employed. For example, the mating surfaces might consist of a series of ribs which would allow the exchangeable nipple element 19 to be “press fit” onto the nipple adapter 17.

The external profile of the nipple element 19 may be one of any number of artificial nipple designs known by those skilled in the art, and is preferably substantially made of silicone or a rubber-type material. A hollowed tip 39 extends from the closed base 33 of the nipple adapter 17 and communicates with the open interior 37. The hollowed tip 39 has an inner and outer diameter, and the outer diameter is sized to be matingly received by the inner diameter of the second extent 31 of the flexible tubing 15.

The mobile breast-feeding assembly is designed so that the nipple adapter 17 may matingly engage several sizes or shapes of exchangeable nipple elements. The nipple adapter 17, as well as each nipple element 19, have matingly sized engageable regions. However, each nipple element 19 can be provided in varying sizes and shapes, thereby allowing the mother to choose her babies favorite sized or shaped nipple.

The hollowed tips 27, 39, respectively, extending from the breast shield 13 and the nipple adapter 17 have specially formed retention surfaces formed thereon which permit the flexible tubing 15 to move in one direction onto the hollowed tips while limiting the movement of the flexible tubing in an opposite direction off the hollowed tips. As seen in FIGS. 2 and 3, in the preferred embodiment of the present invention, the retention surfaces are a series of barbs (such as barbs 41 in FIG. 3).

In the preferred embodiment of the present invention, the mobile breast-feeding assembly described above is adapted for feeding a child while traveling in a motor vehicle. More specifically, the child being fed is seated in a child safety restraint seat. Alternatively, the present invention may be used in any scenario that it is inconvenient or otherwise impossible for a mother to directly breast feed an infant. For example, the assembly would be advantageous for premature infants that are in isolates. The mothers would still be able to directly deliver the milk to the baby while the baby remains in the isolate.

The mobile breast-feeding assembly of the present invention can be made from any convenient material, for example, from a commercially available silicone, rubber, or from a suitable polyolefin, e.g. polypropylene, polyethylene, etc. In one embodiment, it is possible that the present invention could be made disposable, in which case the material might be a latex or similar low cost plastic or rubber. The present invention may be packaged in any of a number of convenient fashions. For example, the present invention may be packaged fully assembled, or conversely, may be packaged as separate components ready to be assembled by the user.

An invention has been provided with several advantages. The breast-feeding assembly provides a secure and convenient way to breast feed a child while traveling in a motor vehicle. The assembly might also be used in situations where the mother is physically isolated from the child, as in the case of premature infants in a hospital setting. The assembly is simple to use and economically viable for all parents. Because the assembly utilizes an exchangeable nipple arrangement, any number of different nipple elements may be utilized, allowing a nursing mother to select a nipple element of a favorite size or shape. Since the device may be provided in a disposable form, it is not necessary to sterilize the various components, making it additionally convenient for use during traveling.

While the invention has been shown in only one of its forms, it is not thus limited, but is susceptible to various changes and modifications without departing from the spirit thereof.

What is claimed is:

1. A mobile breast-feeding assembly, comprising:
a conically shaped breast shield having a base, an apex, and an open interior space;
wherein a hollowed tip extends from the apex and communicates with the open interior space, the hollowed tip having an inner and outer diameter;
a length of flexible tubing having an inner and outer diameter and terminating in first and second opposite extents, wherein the inner diameter of the first extent is sized to matingly receive the outer diameter of the hollowed tip;
a nipple adapter, the adapter having a closed base and cylindrical sidewalls extending outwardly from the base to define an open interior, the sidewalls having external engagement surfaces for matingly engaging an exchangeable nipple element;
wherein a hollowed tip extends from the closed base of the nipple adapter and communicates with the open interior, the hollowed tip having an inner and outer diameter, the outer diameter sized to be matingly received by the inner diameter of the second extent of the flexible tubing;
an exchangeable nipple element engaged on the nipple adapter.

2. A mobile breast-feeding assembly, comprising:
a conically shaped breast shield having a base, an apex, and an open interior space;
wherein a hollowed tip extends from the apex and communicates with the open interior space, the hollowed tip having an inner and outer diameter;
a length of flexible tubing having an inner and outer diameter and terminating in first and second opposite extents, wherein the inner diameter of the first extent is sized to matingly receive the outer diameter of the hollowed tip;
a nipple adapter, the adapter having a closed base and cylindrical sidewalls extending outwardly from the base to define an open interior, the sidewalls being threaded for matingly engaging an exchangeable nipple element;
wherein a hollowed tip extends from the closed base of the nipple adapter and communicates with the open interior, the hollowed tip having an inner and outer diameter, the outer diameter sized to be matingly received by the inner diameter of the second extent of the flexible tubing;
an exchangeable nipple element engaged on the nipple adapter.

3. The mobile breast-feeding assembly of claim 2, wherein the hollowed tips extending from the breast shield and the nipple adapter have specially formed retention surfaces formed thereon which permit the flexible tubing to move in one direction onto the hollowed tips while limiting the movement of the flexible tubing in an opposite direction off the hollowed tips.

4. The mobile breast-feeding assembly of claim 3, wherein the retention surfaces are a series of barbs.

5. The mobile breast-feeding of claim 2, wherein the threaded sidewalls of the nipple adapter are externally threaded and the exchangeable nipple element is internally threaded.

6. A mobile breast-feeding assembly adapted for feeding a child while traveling in a motor vehicle, the apparatus comprising:
a conically shaped breast shield having a base, an apex, and an open interior space;
wherein a hollowed tip extends from the apex and communicates with the open interior space, the hollowed tip having an inner and outer diameter;
a length of flexible tubing having an inner and outer diameter and terminating in first and second opposite extents, wherein the inner diameter of the first extent is sized to matingly receive the outer diameter of the hollowed tip;
a nipple adapter, the adapter having a closed base and cylindrical sidewalls extending outwardly from the base to define an open interior, the sidewalls being threaded for matingly engaging an exchangeable nipple element;
wherein a hollowed tip extends from the closed base of the nipple adapter and communicates with the open interior, the hollowed tip having an inner and outer diameter, the outer diameter sized to be matingly received by the inner diameter of the second extent of the flexible tubing;
an exchangeable nipple element engaged on the nipple adapter.

7. The mobile breast-feeding assembly of claim 6, wherein the hollowed tips extending from the breast shield and the nipple adapter have specially formed retention surfaces formed thereon which permit the flexible tubing to move in one direction onto the hollowed tips while limiting the movement of the flexible tubing in an opposite direction off the hollowed tips.

8. The mobile breast-feeding assembly of claim 7, wherein the retention surfaces are a series of barbs.

9. The mobile breast-feeding assembly of claim 6, wherein the threaded sidewalls of the nipple adapter are externally threaded and the exchangeable nipple element is internally threaded.

10. The mobile breast-feeding assembly of claim 6, wherein the child being fed is seated in a child safety restraint seat.
